(19) United States
(12) Patent Application Publication
(10) Pub. No.: US 2015/0282799 A1
(43) Pub. Date: Oct. 8, 2015

(54) NEURAL TISSUE RETRACTOR SHEATH

(71) Applicant: Shah-Naz Khan, Columbus, GA (US)

(72) Inventor: Shah-Naz Khan, Columbus, GA (US)

(21) Appl. No.: 14/243,966

(22) Filed: Apr. 3, 2014

(51) Int. Cl. A61B 17/02; A61B 1/00

(52) U.S. Cl. CPC A61B 17/0218; A61B 1/00135

(57) ABSTRACT

A moisture-retaining sheath for a medical instrument according to various embodiments can include a surgical retractor and a sheath. The sheath can be configured to include a moisture-retaining layer provided on and covering at least a portion of an outer surface of the surgical retractor for providing a moisture-retaining environment for a tissue site within a treatment worksite identified in a body of a patient when the sheath contacts the tissue site during a surgical procedure.

### I. FIELD OF THE INVENTION

The present disclosure relates generally to a prefabricated or customizable sheath for a medical device. In particular, exemplary embodiments of the present disclosure relates to prefabricated or customizable disposable or reusable sheaths that allow medical devices to be used within the body of a patient without direct tissue contact.

### II. BACKGROUND OF THE INVENTION

Microsurgical techniques are common in intracranial operations, such as for brain tumors, intracranial hematoma, and cerebral aneurysms. In contemporary neurosurgery, during many intracranial procedures, the surgeon works on deeply located structures or brain structures that are required to be separated from each other. Under these circumstances, brain retraction is required for adequate exposure. Retractors have been developed to pull and separate tissue and organs creating space and access for operating.

When a patient undergoes surgery, a surgeon makes an incision in the skin to approach the tissue that requires repair or removal. Often, the body tissue needing repair or removal is positioned below or between a myriad of structures such as veins, arteries, bones, muscles and internal organs. In order to expose an area of targeted tissue within the body, surgeons use retractors to manipulate interposing tissue and hold that interposing tissue out of the way. In this manner, a clear surgical opening can be created to the targeted tissue, thereby enabling a surgeon to observe, repair, or remove, the targeted tissue.

One of the difficulties presented in micro-neurosurgery is that of maintaining effective yet gentle retraction of the brain or the diseased tissue. The operative field is so small that usually there is no space for the surgeon's or assistant's hands. Accordingly, the self-retaining retractor is an important and indispensable instrument for neurosurgery.

Unfortunately, the use of a retractor during brain retraction can cause secondary brain damage. Due to the brain's delicate consistency, such retraction frequently results in trauma to the underlying tissue. Brain retraction puts pressure and may also cause injury to the underlying brain. The pressure of the retractor is transferred to the adjacent tissue, subsequently causing deformation. It may also cause partial or total closure of any underlying blood vessels, resulting in impaired oxygen delivery to brain cells. In order to attenuate such trauma, sometimes various conventional materials, such as, Telfa or cottonoids, are used as a separate and independent intervening layer between the retractor and the tissue in contact with the retractor. This results in the retractor not being directly placed upon the neural tissue itself.

However, there are multiple disadvantages associated with the use of such separate, intervening layer. For example, there is an increase in operating time as each intervening strip is cut to the required size. Another disadvantage is manipulating and appropriately positioning each independent strip. There is also a risk of extensive brain injury if the intervening material is caught in a drill being used in the vicinity of the retractor. Frequently, when the intervening material adheres to the brain during the procedure, the surgeon must remember to irrigate the site appropriately when removing the material to prevent neural injury from adhesion. When the retractor needs to be repositioned during the procedure, the underlying material also needs to be repositioned, or additional strips need to be added, which increases the time and complexity of the procedure.

Oftentimes, the additional time and steps result in surgeons not using any intervening material and tolerating potential trauma to the underlying tissue. Additionally, in an urgent or emergency situation there simply may not be a sufficient amount of time available to undertake these precautionary steps.

Therefore, a device and technique is needed that eliminates the above mentioned shortcomings during neurosurgery, while providing neural tissue protection.

### III. SUMMARY OF THE INVENTION

In at least one aspect, the present disclosure provides a moisture-retaining sheath for a medical instrument including a surgical retractor and a sheath. The sheath can be configured to include a moisture retaining layer provided on and covering at least a portion of an outer surface of the surgical retractor for providing a moisture-retaining environment for a tissue site within a treatment worksite identified in a body of a patient when the sheath contacts the tissue site during a surgical procedure. The sheath can be either prefabricated or customized.

In at least another aspect, the present disclosure provides a method for covering a medical instrument, which includes providing a surgical retractor; and covering at least a portion of an outer surface of the surgical retractor with a sheath having a moisture retaining layer such that a moisture-retaining environment is provided for a tissue site within a treatment worksite identified in a body of a patient when the sheath contacts the tissue site during a surgical procedure. The sheath can be either prefabricated or customized.

In at least one aspect, the present disclosure provides a customizable sheath for a medical instrument. The sheath can be customized by the user during a surgical procedure based on both the treatment worksite and the surgical retractor selected by the user during the surgical procedure. The customized retractor-specific sheath can be configured to have a customized profile defined and shaped by the user during the surgical procedure based on a treatment worksite identified in a body of a patient, such as brain surgery, based on a parameter, such as the removal of a brain tumor or aneurysm clipping during the brain surgery, of the treatment worksite measured in the body of the patient, and based on the selected surgical retractor.

The customized retractor-specific sheath is formed based on the customized profile and applied by the user during the surgical procedure to at least partially enclose the selected surgical retractor and to closely conform to a portion of the outer periphery of the selected surgical retractor such that the customized retractor-specific sheath moves simultaneously with the surgical retractor during the surgical procedure.

In at least another aspect, the present disclosure provides a method for covering a medical instrument, which includes identifying a treatment worksite in a body of a patient; estimating a parameter of the treatment worksite in the body of the patient; selecting a surgical retractor by a user based on at least one of the treatment worksite identified in the body of the patient and the estimated parameter of the treatment worksite; defining and shaping by the user during a surgical procedure a customized retractor-specific sheath having a customized profile based on the treatment worksite identified in the body of the patient, based on the parameter of the treatment worksite measured in the body of the patient, and based on the selected surgical retractor; forming the customized patient-specific sheath based on the customized profile during the surgical procedure; and applying the customized retractor-specific sheath during the surgical procedure to at least partially enclose the selected surgical retractor and to closely conform to a portion of the outer periphery of the selected surgical retractor such that the customized retractor-specific sheath moves simultaneously with the surgical retractor during the surgical procedure.

### IV. BRIEF DESCRIPTION OF THE DRAWINGS

FIG. 1 illustrates a top view of a surgical retractor device enclosed in a sheath in accordance with at least one embodiment of the present disclosure;

FIG. 2 illustrates a side view of the surgical retractor device of FIG. 1;

FIG. 3 illustrates an exploded partial view of the surgical retractor disclosing the sheath in more detail in accordance with at least one embodiment of the present disclosure;

FIG. 4 illustrates an exploded partial view of the surgical retractor disclosing the sheath in more detail in accordance with another embodiment of the present disclosure;

FIG. 5 illustrates a top view of another embodiment of the retractor sheath in accordance with the present disclosure;

FIG. 6 illustrates a top view of yet another embodiment of the retractor sheath in accordance with the present disclosure;

FIG. 7 illustrates a top view of another embodiment of the sheath partially enclosing portions of the surgical retractor in accordance with the present disclosure;

FIG. 8 illustrates a side view of the surgical retractor in FIG. 7;

FIG. 9A illustrates a side view of another embodiment of the surgical retractor enclosed within the sheath in accordance with the present disclosure;

FIG. 9B illustrates a side view of a further embodiment of the surgical retractor enclosed within the sheath in accordance with the present disclosure;

FIG. 10 is a flowchart of an exemplary method of practicing an embodiment of the present disclosure; and

FIG. 11 is a flowchart of another exemplary method of practicing an embodiment of the present disclosure.

The present disclosure may take form in various components and arrangements of components, and in various process operations and arrangements of process operations. The present disclosure is illustrated in the accompanying drawings, throughout which, like reference numerals may indicate corresponding or similar parts in the various figures. The drawings are only for purposes of illustrating preferred embodiments and are not to be construed as limiting the disclosure. Given the following enabling description of the drawings, the novel aspects of the present disclosure should become evident to a person of ordinary skill in the art.

### V. DETAILED DESCRIPTION OF THE DRAWINGS

The following detailed description is merely exemplary in nature and is not intended to limit the applications and uses disclosed herein. Further, there is no intention to be bound by any theory presented in the preceding background or summary or the following detailed description. While embodiments of the present technology are described herein primarily in connection with neural tissue retractor sheaths, the concepts are also applicable to other types of sheaths and removal and repair of various body tissues.

Many surgical procedures are standardized. Therefore, surgeons can anticipate the type of interposing tissue that will be encountered in different types of surgical procedures. As a result, retractors are manufactured in a variety of different configurations in order to be useful for the different surgical procedures. Most specialized retractors are made of a predetermined shape and size in view of the intended use. Hundreds of different types of surgical retractors exist. When a surgical team operates, they select the retractors that they will most probably need.

Malleable retractors have been developed to address these improvised situations. Malleable retractors are retractors that are usually long, thin and flat. The retractors are made from malleable material that can be custom bent into most any desired shape during the surgery. As such, surgeons can be assured that they can adapt the malleable retractor to their needs during the surgery. Malleable retractors are more versatile than rigid stainless steel retractors used for other parts of the body.

Various embodiments of the present disclosure relate to a sleeve, cover or a sheath that is slipped over malleable retractors. The portion of the sheath in contact with brain or similar soft tissue is made of non-adherent material such as, but not limited to, polyethylene terephthalate. The portion of the sheath not in contact with the soft tissue may also be made of the same or alternative material and may be non-glaring under the microscope's luminescence.

Malleable retractors are available in various sizes. Therefore, the present disclosure relates to sheaths that can be conformed to various sizes by selectively varying the configuration to cover multiple types of retractors. In various embodiments, the sheath or cover is supplied preassembled or can be easily cut, torn, folded, or adhered together to further modify and customize its size, as required. Various embodiments of the sheath is configured to closely conform with the size of the retractor such that there is no excess extending to potentially be caught in operative devices, such as drills, or cause unwelcome glare under a microscope. In various embodiments, the sheath can be customized by the user to a size so that the malleable retractor can continue to be modified in its curvature as needed, such that the sheath does not interfere with the attachment of the retractor into its retaining mechanism. In various embodiments, this customization may be achieved by either cutting the end of the sheath adjacent to the attachment site to make it shorter, or by providing a slit(s).

Therefore, various embodiments provide a standard configuration that permits custom applications to form different shapes and sizes to fit various retractors. The construction of the sheath can be easily modified for custom applications while allowing for standardization of a product. The sheath can be selected from a number of standard, pre-sized and pre-shaped configurations by merely varying the length of the sheath to provide an apparatus having a selected desired configuration. Thus, the sheath provides a simple device that can be easily configured to cover various sized retractors. The system provides a means of using the same sheath for several different applications and size requirements, whereby to enable the user to customize the sheath to the particular requirements. In various embodiments, by merely changing the length and/or width, the sheath is easily altered or modified to fit the desired size and application.

The sheath described in the present disclosure offers many advantages. For example, no time is lost during the operating procedure by preparing several individual strips of required sizes. As the sheath is slipped onto the retractor itself, the sheath travels with the retractor such that no time is lost in transferring and positioning several protective strips. Thus, there is no additional time added to the procedure for repositioning several protective strips and other protective maneuvers. In various embodiments, the retractor sheath is moistened, and the moist retractor sheath will also keep the underlying brain tissue moist, as is desirable. The ability to customize the sheath to the correct size allows for the malleable retractor to be molded into various shapes without difficulty. The customization of the sheath also prevents excess strip from extending beyond the retractor to interfere with surgical instruments.

In other various embodiments, the protective material is configured in the form of a sheet, rather than a sleeve or sheath. In various embodiments, the sheet may include an adhesive strip at one or more ends whereby it can be folded or, conversely, wrapped around the retractor to create a ‘custom’ sleeve by means of the adhesive strips.

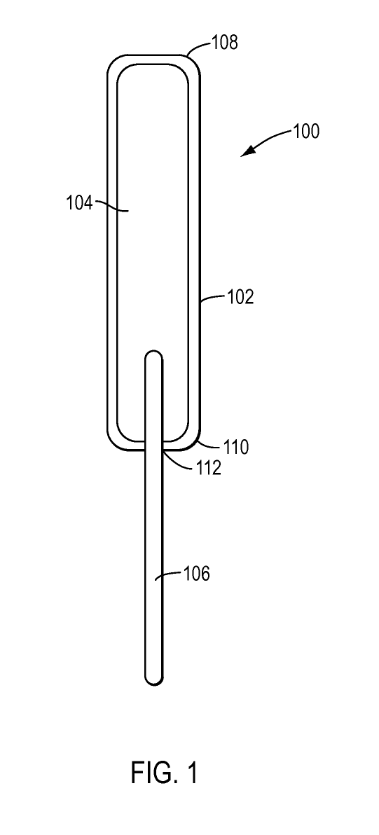

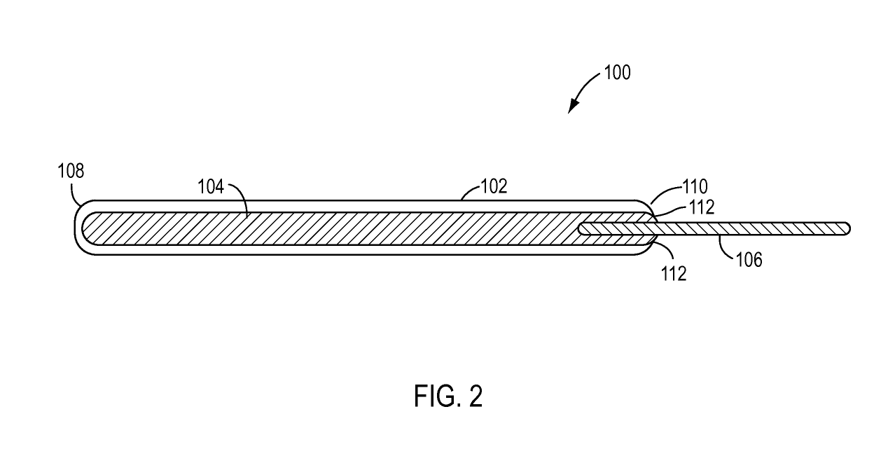

An exemplary embodiment of a malleable retractor 100 that can be used to assist with removing, positioning or holding tissue or organs during a surgical procedure is illustrated in FIGS. 1-2. For the purpose of this disclosure, the purposely generalized term “tissue” will be taken to include human or animal flesh (including skin, fat, muscle, vascular, tumor and nerve tissues). Surgical procedures often require that tissues and organs be temporarily displaced to provide a clear visual path for the surgeon. The arterial and venous systems of a human being is often covered or obscured by body tissues and/or organs that may need to be repositioned during a surgical procedure. The obscuring tissues and/or organs may be brain tissue, which may need to be retracted and held in a preferred position away from the point of surgery. FIGS. 1-2 illustrate a retraction device 100 of the present disclosure that may be placed in a surgical area, for example such as the brain, to provide an unobstructed workplace. The retraction device 100 operates to retract or reposition tissue of the brain away from the immediate workplace. Once the retraction device 100 is properly placed, the surgeon will have a clear and reasonably unobstructed view of the operative site.

The retraction device 100 in the exemplary embodiments of the disclosed figures depicts a soft tissue malleable retractor for use, for example, during the process of dissecting the brain during brain surgery. An exemplary soft tissue retractor 100 is shown which can include a deformable blade member 104 (also commonly referred to as a “spatula” or a “plate”) having a proximal end and a distal end. The retractor 100 further includes a handle element 106, which is affixed to the proximal end of the blade member 104. The soft tissue retractor 100 can include a sheath 102 for enveloping and receiving at least a portion of the blade 104 that contacts body tissue during the surgical procedure.

The term “deformable” should be understood to mean consisting of a pliable and inherently shape-retaining material, which is sufficiently malleable that it can be bent to any configuration by hand and retains its shape after bending. Because it is malleable, such retractors may be bent into shapes that are suitable for various surgical procedures. A malleable retractor may be constructed in a variety of ways. For example, the malleable retractor may be formed from a malleable metal such as annealed stainless steel, copper, titanium, soft malleable iron or aluminum, or other suitably deformable metal and alloys. Malleable plastics or any other suitable material may also be employed in various embodiments.

Retractors other than soft tissue retractors may be used according to the present disclosure for tissue retraction, removal, cushioning, or positioning during a surgical procedure. Those skilled in the art would recognize that a variety of retractors may be used in accordance with the present teaching. It is understood that the retractor 100 shown herein is merely one of many conventional forms currently in use. It should be further understood, however, that the present sheath 102 is configured to be sufficiently resilient and pliable to fit over many blade configurations. Furthermore, the sheath 102 can be produced with varying shapes and sizes to conform to virtually any retractor blade structure. For example, the malleable retractor may be any commercially available retractor, hand held retractor or self-retaining retractor attachable to retraction bars, halos and other devices.

As shown in FIGS. 1-2, the retractor 100 includes a customizable sheath 102 having a closed distal end 108 and an open proximal end 110 forming a cover generally in conformity with the curvature of the blade member 104. The terms “proximal” and “distal” are used herein to refer to the relative positions of the retractor 100. As used herein, “proximal” refers to a position relatively closer to the exterior of the body or closer to a user (such as, a surgeon, technician, nurse, etc.) of the retractor 100. In contrast, “distal” refers to a position relatively further away from the user of the retractor 100 or closer to the interior of the body.

FIG. 2 depicts an exemplary sheath 102 of the current disclosure. Sheath 102 may include a flexible cover that envelopes blade member 104 and extends between the proximal end 110 and the distal end 108. During application, the distal end 108 may be positioned within the body of a patient while the proximal end 110 may extend outside the body. The distal end 108 of the sheath 102 may be inserted into the body of the patient and pushed into a body cavity (such as, for example, in the brain) to position the distal end 108 at a desired work site within the body.

The sheath 102 can be made from a flexible, elastic and resilient material, which can intimately conform to the shape of the blade member 104. In various embodiments, the exterior surface of the sheath can be smooth, meshed, irregular, or textured. The sheath cover 102 is compatible for insertion within the body. Possible materials for the sheath 102 may include but are not limited to material, such as, for example, thermoplastic, polymers, polyethylene terephthalate (PET), polyurethanes, polyethylenes, ionomers, copolyesters, silicon polymers, rubbers, polyamides, plastics, any other suitable materials known in the art or mixtures thereof.

Further, the sheath cover 102 may include an antiglare layer, material or color, so that the cover is non-glaring under microscope luminescence. Surgery is usually performed with bright operating room lights directed downwardly onto the surgical site during surgical procedures. Surgical microscopes also include internal lighting directed through the objective lens. The lighting can result in glare, making it more difficult for the surgeon to see the surgical site. To reduce glare, the sheath 102 is provided with an antiglare layer or texture, for example, to direct the reflected light away from the lens of the microscope. The antiglare property of the sheath 102 will make the surgical site be seen more clearly through the microscope.

FIG. 2 illustrates an example of the coverage of the blade 104 of the malleable retractor 100 by the cover of the sheath 102. Sheath 102 may be affixed to the retractor 100 in any manner. In some embodiments, the sheath 102 may be slid over the retractor 100. It is also contemplated, in various embodiments, that the sheath 102 may, for example, be rolled over the retractor 100, wrapped, or adhered by adhesives.

The sheath 102 envelopes at least a part of the blade 104 which is intended to make contact with the body tissue, such as the brain tissue. In the preferred embodiment, the sheath envelopes the entire blade member 104. In another embodiment, the sheath 102 covers only the portion of the retractor 100 which makes contact with the body tissue. In both embodiments, this results in the cover material of the sheath 102 functioning as an intervening layer between the body tissue and the retractor 100 to protect the soft, fragile tissue of the brain.

In various embodiments, the material of the sheath 102 is non-adherent to soft tissue, such as brain tissue, in order that the sheath serves as an interface between the body tissue and the retractor to protect the underlying delicate tissue. For example, the material of the sheath may be a polyethylene, such as polyethylene terephthalate. The non-adherent property also enables the sheath to act as a liner for the delicate tissue assuring easy removal of the sheath simultaneously with the displacement of the retractor 100. Thus, the sheath 102 will also ‘lift’ and move with the retractor 100, whenever it is so displaced, without sticking to, damaging, cutting or tearing the soft tissue. The sheath 102 may also be non-linting. Thus, the cover of the sheath moves simultaneously along with the ensheathed retractor, enabling efficient and safer tissue retraction.

Due to its composition, in various embodiments, the sheath may be fluid retaining. In most cases, no more than a small amount of fluid flow will be needed to irrigate a work site within the body. In such embodiments, the material of the sheath may remain moist for an extended duration; thereby, also keeping the underlying brain surface moist to facilitate a smooth transition of re-positioning the covered retractor during the surgical procedure.

In other embodiments, the sheath can be used to seal, cover and/or protect various parts of the retractor system. The sheath can beneficially be used to reduce contamination by protecting the retractor 100 from contamination when used in various parts of the body. The sheath 102 can surround the retractor 100 and seal the interface between the blade member 104 and the handle 106. For example, in various embodiments, the proximal end 110 of the sheath 102 is configured to cover and create a fluid tight seal 112 around a connection between the blade member 104 and the handle 106 such that the sheath 102 completely encloses the blade member 104 as shown in FIG. 2. Thus, when the sheath 102 shown in FIGS. 1-2 is applied to the retractor 100, the retractor 100 is inserted through the open proximal end 110 through the sealing element 112, whereby the sealing element 112 bears compressively against the base of the retractor 100.

Sheath 102 may be either a disposable sheath or a reusable sheath. If sheath 102 is a reusable sheath, it should be made from materials easily cleanable and sterilizable. If sheath 102 is disposable, it should be an easily replaceable component of the retractor system and may be made from any suitable biocompatible material, such as a plastic, PET or other polymer.

In some embodiments, the sheath 102 may be only partially disposable with a remainder of the device being reusable. In these embodiments, a portion of the sheath that contacts body tissue may be a disposable portion. The disposable portion may mate at an interface (not shown) with a reusable portion. The disposable and reusable portions together form the sheath. The interface may also be configured to prevent biological tissue from contacting the reusable portion. Any type of interface that enables the disposable and reusable portions to be coupled together while isolating the reusable portion from the body tissue may be used. The interface may be an integral part of one of the portions (such as, for example, an integral part of the disposable portion). In some medical procedures using a sheath with disposable and reusable portions, the two portions may be first coupled together before the sheath is inserted into the body. In other embodiments, only one of the portions (such as for example, the disposable portion which contacts the human tissue) may be coupled to the retractor 100 before the sheath 102 is inserted into to the body. The remaining portion (such as, for example, the reusable portion) may be coupled after the sheath 102 is inserted into to the body.

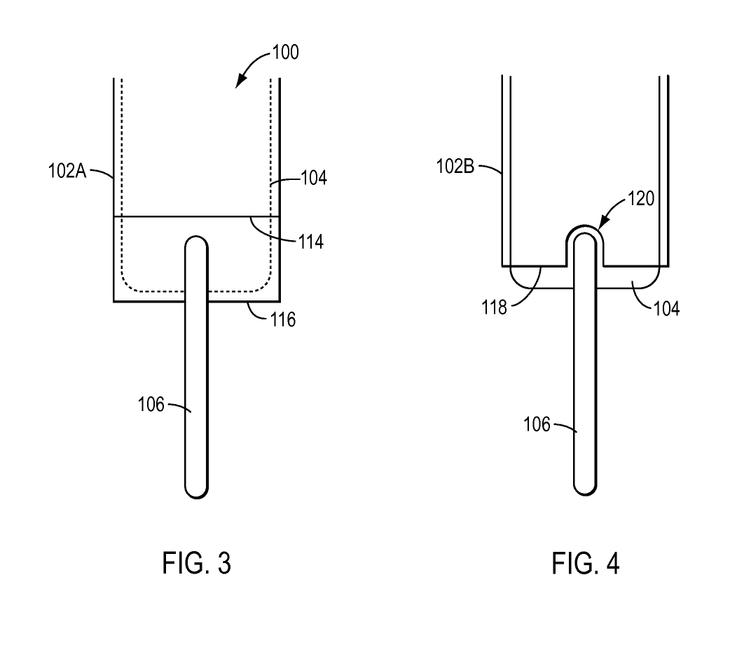

FIG. 3 is an expanded view of FIG. 1 depicting an exemplary embodiment of the handle 106 or hand holding area attached to the blade 104 of the retractor 100. The blade member 104, shown with broken lines in FIG. 3, is ensheathed by the sheath 102A. The handle 106 is sufficiently long to extend beyond the sheath 102A after the blade 104 is slidably and snugly fit within the sheath 102A. The handle 106 of the retractor 100 extends out of the sheath for easy grasping, thereby facilitating usage of the retractor 100 by the user.

In various embodiments, the handle 106 may be a permanent and integral part of the retractor system 100. The handle 106 may be permanently connected to the blade 104 through the use of instrumentalities, such as adhesives, weld(s) and/or screws or other mechanical fasteners, that do not allow the handle to be removed without disassembly or destruction of at least a portion of the retractor system. In other embodiments, the handle 106 may be releasably connected to the blade member 104, for example, through the use of a quick-connection. In various embodiments, the handle may be attached via a retractor arm to a Halo, attachment bar, or similar system, making the retractor ‘self-retaining.’

In various embodiments, the handle 106 may include controls (not shown) that may activate different features of the retractor 100. The retractor 100 may include one or more bendable sections positioned along its length. In some embodiments, these bendable sections may be configured to bend or deflect in response to the actuation of the controls. These controls may actuate the bendable sections in any manner. In some embodiments, these controls may, mechanically, thermally, or electrically actuate the bendable sections to deflect in a desired direction.

In various embodiments, the blade member 104 and the handle 106 may be arranged to be coupled together so that the handle 106 projects from the proximal end 110 of the sheath 102 and the handle 106 can be rotated about with respect to the longitudinal axes.

To use the retractor 100, the handle 106 can be either manually or electronically deformed so as to position the blade member 104 where it is needed during a surgical procedure. Accordingly, the handle 106 can be selectively bent by a surgeon, during an operation, so as to properly orient the blade member 104.

According to the present teachings, the sheath may be configured having many different shapes and different sizes depending on the retractor to be contained therein. In various embodiments, the sheath can be customized by the user to a size to fit the curvature of the retractor, such that the sheath does not interfere with the attachment of the handle 106 or other components into their retaining mechanisms. In various embodiments, this customization may be achieved, for example, by cutting, folding or adhering the end of the sheath or customizable sheath material to confirm with a particular blade, or by providing one or more slits. The slits function as an accommodation feature for the retractor construction, for example, at the junction of the handle and blade, or other design features of a particular blade. The ability to customize the sheath to the correct size allows for the malleable retractor to be molded into various shapes without difficulty. The customization of the sheath also enables the sheath to closely conform to the size of the retractor such that there is no excess extending beyond the retractor to potentially be caught in operative devices.

FIGS. 3-8 illustrate various embodiments of varying the length and/or configuration of the sheath 102 according to the present teachings. In various embodiments, sheath 102 is constructed to be longitudinally expandable by stretching the material of the sheath or by unfolding or unrolling the sheath. By manipulating the length of the sheath 102, the proximal end 110 (shown in FIGS. 1-2) can be formed into an entire range of lengths. The sheath 102 can be formed having a variety of lengths and widths. Having a sheath that can be modified on site to form a particular length according to the present teaching, increases surgical efficiency. Therefore, the present disclosure relates to sheaths that can be conformed to various sizes by selectively varying the configuration to cover multiple types of retractors based on the surgical worksite. In various embodiments, the sheath or cover can be easily cut, torn, folded, or adhered together to further modify its size, as required.

FIG. 3 illustrates a sheath 102A having variable lengths as configured by the user during the surgical procedure. In this example, the top edge 114 and bottom edge 116 of the sheath 102A are configured having different lengths. The top edge 114 covers substantially the front of the blade member 104, and the bottom edge 116 covers the entire back of the blade member 104. In one embodiment, the user may cut the top edge 114 and the bottom edge 116 such that they have variable lengths. In this example, the top edge 114 and the bottom edge 116 may both remain unsealed.

In lieu of being cut by the user and remaining unsealed, the top edge 114 and the bottom edge 116 of the sheath 102A may include a combination of perforations and adhesive to provide a sealed sheath 102A. In this example, selected locations of the sheath 102A may include one or more lines of weakness that function as perforation lines that can be selectively torn off, for example, at top edge 114 and bottom edge 116. The lines of weakness may be regions where the strength is intentionally reduced so that the sheath 102A may fold or tear along the line. Sheath 102A may include many different types of lines of weakness. In some embodiments, such lines of weakness include a fold line and/or perforation lines. Fold line may be a line along which the strength of the sheath 102A is reduced so that the sheath may fold along the fold line when a bending force is applied to the sheath. As used herein, bending force merely refers to a force applied to the sheath that may bend sheath along a fold line.

The sheath may also include many different types of perforation lines. Perforation lines may include lines along which the strength of the sheath is reduced so that the sheath may tear along this line when a tearing force is applied to sheath. As used herein, tearing force merely refers to a force applied to the sheath that may tear the sheath along the perforation line. The fold lines and the perforation lines may be constructed by conventional techniques.

The inside surface of sheath 102A may include an adhesive material, such as adhesive strips. Adhesive strips may include any type of adhesives or glues known in the art. In some embodiments, the sheath 102A may include a layer of adhesive attaching different sections of the sheath 102A to the blade member 104. In some embodiments, sheath 102A may include one or more adhesive strips. These adhesive strips may include a layer of sticky material covered with a layer of covering material.

To temporarily attach the sheath 102A to the blade member 104, in embodiments employing the adhesive strips, the desired length of the top edge 114 and the bottom edge 116 can be selected and torn based on the perforated lines. The top edge 114 and the bottom edge 116 may then be temporarily sealed to the blade member 104 by removing the covering material from the adhesive strips and attaching the sheath 102A together with the blade member 104 using the layer of the sticky material.

FIG. 4 illustrates another embodiment of the sheath 102B having a lower edge 118 comprising a substantially uniform length and including a cut-out 120 that surrounds a portion of an attachment point of the handle 106. Similar to FIG. 3, the lower edge 118 may be selectively cut by the user during a surgical procedure. Alternatively, the lower edge 118 may be configured having a sealed sheath as described above with reference to FIG. 3.

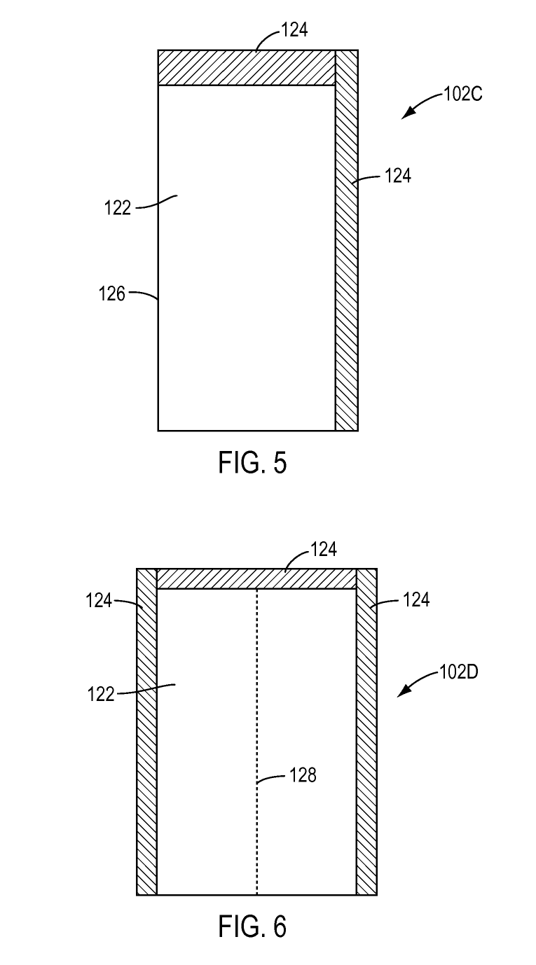

FIG. 5 depicts an alternative embodiment wherein the cover material is in the form of a sheet 122 provided with one or more adhesive strips 124. The sheet 122 can be folded to create a sheath 102C having a desired width and length. In the disclosed embodiment, the sheet 122 is provided with two adhesive strips 124 that function as sealing edges, at the top and side thereof, respectively, defining the sheath 102C. Sheath 102C is created by removing adhesive strips 124 to expose the adhesive material on the edges and then folding the sheet 122 parallel to its greatest length along folding guide line 126. In this fashion, a folded sheet assembly having a closed distal end and an open proximal end is provided. The periphery of the top and side of the sheet is provided with an adhesive surface area covered by removable strips to permit the sheath 102C to be folded onto itself and adhesively secured together to enclose the blade within the sheath. It will be readily apparent that in lieu of the adhesive strips, any suitable adhesive coating, tapes, and the like can be used.

In lieu of folding guide lines 126 as shown in FIG. 5, FIG. 6 depicts an exemplary embodiment wherein the cover material is in the form of a sheet 122 provided with an adhesive material, such as one or more adhesive strips 124, and having one or more cutting guide lines 128, which can be used to create a sheath 102D having a desired width and length. In this example, sheath 102D is configured to have a surface including indicia for demarking one or more guide lines 128 for cutting. The predetermined guide lines 128 define cutting tracks formed, for example, as perforation lines by lines of weakness that can be easily cut. The markings of the guide lines can function as lines of reference with respect to the type, size or shape of the retractor. In this embodiment, the medical professional can select any guide line for cutting the sheath 102D to the desired width and length based on the retractor to be contained therein. Rather than including a cutting guide line, some embodiments may only include a visual guide line.

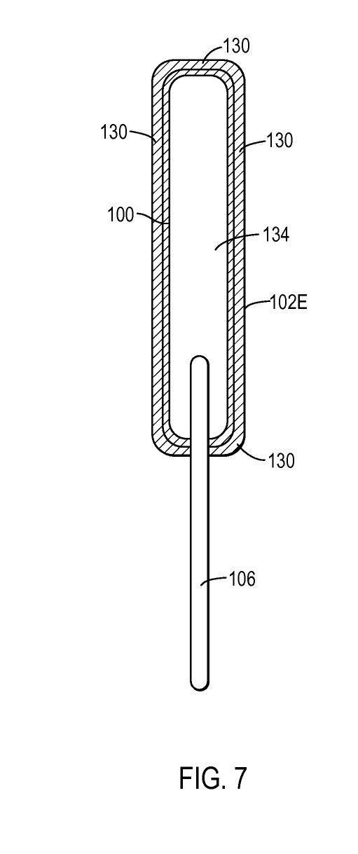

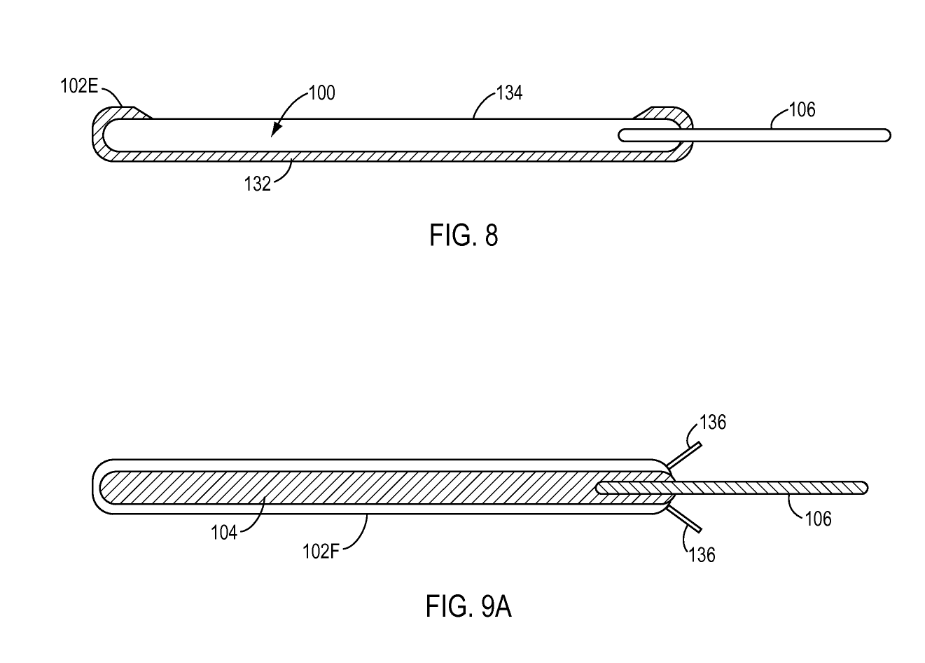

FIGS. 7-8 illustrate an alternative embodiment where the sheath 102E covers the outer edges 130 and a tissue-contacting surface 132 of the retractor 100, which only makes contact with the body tissue, such as the brain tissue. In this example, the non-tissue contacting surface 134 (FIG. 8) remains bare and uncovered. The contact surface 132 of the malleable retractor 100 is positioned adjacent to the body tissue, covered by sheath 102E. In this example, the sheath 102E also covers the outer edges 130 of the retractor 102E.

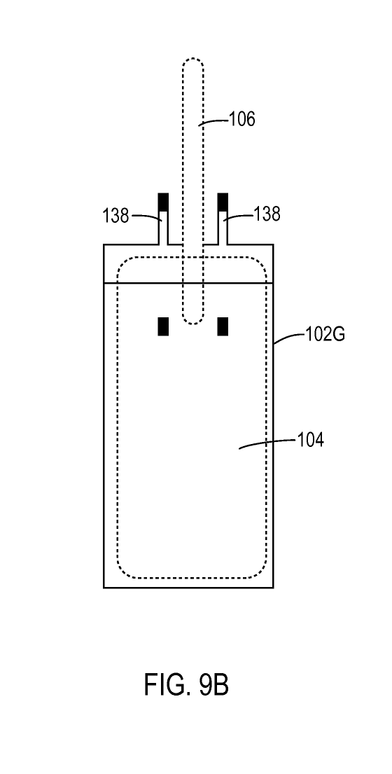

FIG. 9A illustrate an embodiment of the sheath 102F including one or more pull tabs or pull flaps 136, which provide extended surface elements for manual gripping to assist with applying the sheath to the blade member 104. The pull tabs or flaps 136 can be used by the medical professional to assist with quickly applying the closely conforming sheath onto the blade member 104. After the sheath 102F is applied to the blade member 104, the user can easily remove the pull tabs or flaps 134 by tearing them off and then discharging them.

FIG. 9B illustrate an embodiment of the sheath 102G including one or more pull tabs or pull flaps 138, which provide extended surface elements for manual gripping to assist with applying the sheath to the blade 104. The pull tabs or flaps 138 can be used by the medical professional to assist with quickly applying the closely conforming sheath onto the blade member 104. In this embodiment, after the sheath 102G is applied to the blade member 104, the pull tabs 138 can be folded over the retractor and adhered to the front surface of the sheath 102G.

In another embodiment of the invention, the adhesive surface is attached to only the part of the blade that contacts the tissue, such as brain, during retraction. Such strips can be prefabricated in various sizes, or custom cut from provided material as needed.

In another embodiment of the invention, the material is permanently attached to the retractor. In such case, the material may be, for example, silicone gel, but not restricted to silicone gel in other embodiments.

In general, the present disclosure relates to a pre-fabricated or customizable sheath for a medical instrument. The sheath can be customized by the user during a surgical procedure based on both the treatment worksite and the surgical retractor selected by the user during the surgical procedure. The customized patient-specific sheath can be configured to have a customized profile defined and shaped by the user during the surgical procedure based on a treatment worksite identified in a body of a patient, such as brain surgery, based on a parameter, such as the removal of a brain tumor during the brain surgery, of the treatment worksite measured in the body of the patient, and based on the selected surgical retractor.

The customized retractor-specific sheath is formed based on the customized profile and applied by the user during the surgical procedure to at least partially enclose the selected surgical retractor and to closely conform to a portion of the outer periphery of the selected surgical retractor such that the pre-fabricated or customized retractor-specific sheath moves simultaneously with the surgical retractor during a surgical procedure.

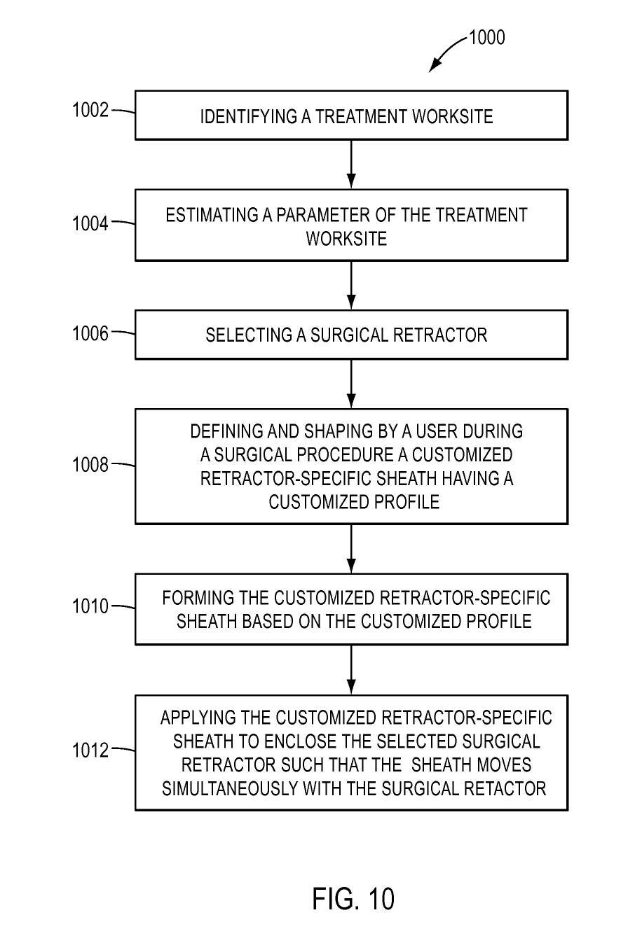

FIG. 10 is a flow chart that illustrates an exemplary method of using a sheath in accordance with the present disclosure. In the exemplary method 1000, the user identifies a treatment worksite in a body of a patient during a surgical procedure, such as brain surgery in step 1002. In step 1004, the user measures a parameter of the treatment worksite, such as the removal of a brain tumor during the brain surgery. In step 1006, the user selects a surgical retractor, for example, based on treatment worksite and the measured parameter. In step 1008, the user defines and shapes during the surgical procedure a customized retractor-specific sheath having a customized profile based on treatment worksite identified in the body of the patient, based on the parameter of the treatment worksite measured in the body of the patient, and based on the selected surgical retractor. In step 1010, the user forms the customized patient-specific sheath based on the customized profile during the surgical procedure. In step 1012, the user applies the customized retractor-specific sheath during the surgical procedure to at least partially enclose the selected surgical retractor and to closely conform to a portion of the outer periphery of the selected surgical retractor such that the customized retractor-specific sheath moves simultaneously with the surgical retractor during the surgical procedure.

In various embodiments, the sheath is composed of moisture-retaining material that will not adhere to tissue and can be easily separated from underlying tissue. The material of the sheath also should be malleable to adapt to the changing shapes of the retractor. Therefore, Telfa or similar material may be used for this purpose. Thus, the medical professional may cut or customize the sheath to a size that covers the selected retractor during the surgical procedure. The sheath having a non-adherent property is slipped onto the malleable retractor. The sheath may then be moistened. The retractor covered by the sheath is then used to retract the underlying tissue. During the surgical procedure, the retractor can be repositioned when needed and the sheath will move along with the retractor, without requiring separate repositioning. Using material that is non-adherent to the brain or other tissue enables the sheath to move simultaneously along with the ensheathed retractor, enabling efficient and safer tissue retraction.

In some embodiments, a system and method for a moisture-retaining sheath for a medical instrument is provided. The moisture-retaining sheath may include a surgical retractor and a sheath. The sheath may include a moisture-retaining layer provided on and covering at least a portion of an outer surface of the surgical retractor. Overlaying the sheath on the surgical retractor creates a moisture-retaining environment for a tissue site within a treatment worksite identified in a body of a patient when the sheath contacts the tissue site during a surgical procedure. In this embodiment, the sheath moves simultaneously with the surgical retractor during the surgical procedure.

The moisture-retaining sheath is configured as a tissue-interface layer for contacting the tissue site within the treatment worksite. The tissue-interface layer is non-adherent to the tissue site when the sheath is removed from contact with the tissue site.

In some embodiments, the moisture-retaining sheath is prefabricated. In other embodiments, the moisture-retaining sheath is customized, assembled, and attached to the surgical retractor during the surgical procedure, as described above. In the customized embodiment of the moisture-retaining sheath, the sheath may include perforations, indentions, adhesive, cutting guide lines, folding guide lines and/or markings to enable the sheath to be customized for different surgical retractors.

The moisture-retaining sheath may also include an antiglare coating adhered to a surface of the sheath. The antiglare coating reduces glare transmitted from a microscope and transmitted from lighting sources within an operating room where the surgical procedure is performed, to enhance visibility of the worksite during the surgical procedure. The moisture-retaining sheath may comprise a layer selected from the group consisting of thermoplastic polymers polyethylene terephthalate (PET), polyurethanes, polyethylenes and ionomers, copolyesters, silicon polymers, rubbers, polyamides, plastics, or combinations thereof.

In various embodiments, surgical retractor of the moisture-retaining sheath includes a blade member attached to a handle. The sheath comprises a sealing element configured to form a substantially fluid tight seal between the blade member and the handle to prevent fluid flow communication between the surgical retractor and an environment external to the surgical retractor. In various embodiments, the sheath is formed having a profile configured to enclose only a tissue-contacting surface area of the surgical retractor during the surgical procedure.

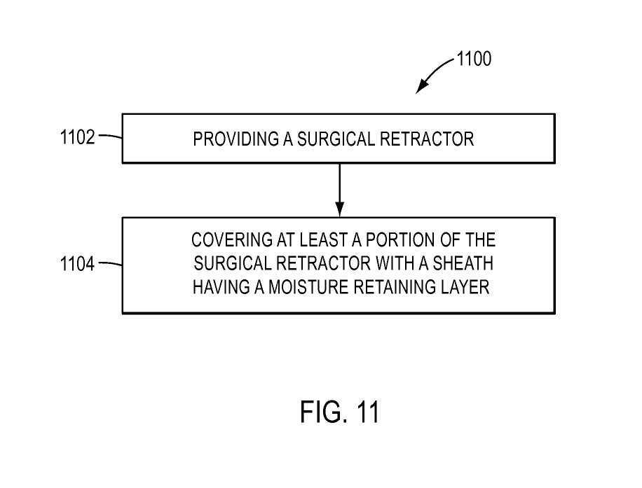

FIG. 11 is a flow chart that illustrates an exemplary method of using a moisture-retaining sheath in accordance with the present disclosure. In the exemplary method 1100, a surgical retractor is provided in step 1102. In step 1104, at least a portion of the surgical retractor is covered with a sheath having a moisture-retaining layer such that a moisture-retaining environment is provided for a tissue site within a treatment worksite identified in a body of a patient when the sheath contacts the tissue site during a surgical procedure.

Those skilled in the art will also appreciate that various adaptations and modifications of the preferred and alternative embodiments described above can be configured without departing from the scope and spirit of the disclosure. Therefore, it is to be understood that, within the scope of the appended claims, the disclosure may be practiced other than as specifically described herein.

We claim:

1. A moisture-retaining sheath for a medical instrument, comprising:
a surgical retractor; and
a sheath having a moisture-retaining layer provided on and covering at least a portion of an outer surface of the surgical retractor for providing a moisture-retaining environment for a tissue site within a treatment worksite identified in a body of a patient when the sheath contacts the tissue site during a surgical procedure.

2. The sheath according to claim 1, wherein the sheath moves simultaneously with the surgical retractor during the surgical procedure.

3. The sheath according to claim 1, wherein the sheath is configured as a tissue-interface layer for contacting the tissue site within the treatment worksite and the tissue-interface layer is non-adherent to the tissue site when the sheath is removed from contact with the tissue site

4. The sheath according to claim 1, wherein the sheath is prefabricated.

5. The sheath according to claim 1, wherein the sheath is customized, assembled, and attached to the surgical retractor during the surgical procedure.

6. The sheath according to claim 5, wherein the sheath includes perforations, indentions, adhesive, cutting guide lines, folding guide lines and/or markings to enable the sheath to be customized for different surgical retractors.

7. The sheath according to claim 1, further comprising an antiglare coating adhered to a surface of the sheath, the antiglare coating reduces glare transmitted from a microscope and transmitted from lighting sources within a room where the surgical procedure is performed, to enhance visibility of the worksite during the surgical procedure.

8. The sheath according to claim 1, wherein the sheath comprises a layer selected from the group consisting of thermoplastic polymers polyethylene terephthalate (PET), polyurethanes, polyethylenes and ionomers, copolyesters, silicon polymers, rubbers, polyamides, plastics, or combinations thereof.

9. The sheath according to claim 1, wherein the surgical retractor includes a blade member attached to a handle, and
wherein the sheath comprises a sealing element configured to form a substantially fluid tight seal between the blade member and the handle to prevent fluid flow communication between the surgical retractor and an environment external to the surgical retractor.

10. The sheath according to claim 1, wherein the sheath is formed having a profile configured to enclose only a tissue-contacting surface area of the surgical retractor during the surgical procedure.

11. A method for covering and protecting a medical instrument, comprising:
providing a surgical retractor;
covering at least a portion of an outer surface of the surgical retractor with a sheath having a moisture-retaining layer such that a moisture-retaining environment is provided for a tissue site within a treatment worksite identified in a body of a patient when the sheath contacts the tissue site during a surgical procedure.

12. The method according to claim 11, further comprising moving simultaneously the sheath with the surgical retractor during the surgical procedure.

13. The method according to claim 11, wherein the sheath is configured as a tissue-interface layer for contacting the tissue site within the treatment worksite and the tissue-interface layer is non-adherent to the tissue site when the sheath is removed from contact with the tissue site

14. The method according to claim 11, wherein the sheath is customized, assembled, and attached to the surgical retractor during the surgical procedure.

15. The method according to claim 14, wherein the sheath includes perforations, indentions, adhesive, cutting guide lines, folding guide lines and/or markings to enable the sheath to be customized for different surgical retractors.

16. The method according to claim 11, further providing an antiglare coating adhered to a surface of the sheath, the antiglare coating reduces glare transmitted from a microscope and transmitted from lighting sources within a room where the surgical procedure is performed, to enhance visibility of the worksite during the surgical procedure.

17. The method according to claim 11, wherein the sheath comprises a layer selected from the group consisting of thermoplastic polymers polyethylene terephthalate (PET), polyurethanes, polyethylenes and ionomers, copolyesters, silicon polymers, rubbers, polyamides, plastics, or combinations thereof.

18. The method according to claim 11, wherein the surgical retractor includes a blade member attached to a handle, and
wherein the sheath comprises a sealing element configured to form a substantially fluid tight seal between the blade member and the handle to prevent fluid flow communication between the surgical retractor and an environment external to the surgical retractor.

19. The method according to claim 1, wherein the sheath is formed having a profile configured to enclose only a tissue-contacting surface area of the surgical retractor during the surgical procedure.

20. A sheath for a medical instrument, comprising:
a surgical retractor selected by a user during a surgical procedure;
a customized retractor-specific sheath having a customized profile defined and shaped by the user during the surgical procedure based on a treatment worksite identified in a body of a patient, based on a parameter of the treatment worksite estimated in the body of the patient, and based on the selected surgical retractor; and
the customized retractor-specific sheath is formed based on the customized profile and applied by the user during the surgical procedure to at least partially enclose the selected surgical retractor and to closely conform to a portion of the outer periphery of the selected surgical retractor such that the customized patient-specific sheath moves simultaneously with the surgical retractor during the surgical procedure.
